(19) United States
(12) Patent Application Publication
(10) Pub. No.: US 2022/0071780 A1
(43) Pub. Date: Mar. 10, 2022

(54) METHOD FOR CONTROLLING AN ORTHOTIC OR PROSTHETIC DEVICE AND ORTHOTIC OR PROSTHETIC DEVICE

(71) Applicant: OTTO BOCK HEALTHCARE PRODUCTS GMBH, Wien (AT)

(72) Inventors: Dirk SEIFERT, Wien (AT); Juan Pablo MEJIA NINO, Mödling (AT)

(73) Assignee: OTTO BOCK HEALTHCARE PRODUCTS GMBH, Wien (AT)

(21) Appl. No.: 17/423,834

(22) PCT Filed: Jan. 16, 2020
(86) PCT No.: PCT/EP2020/051003

§ 371 Date: Jul. 16, 2021

(30) Foreign Application Priority Data
Jan. 17, 2019 (DE) 10-2019 101 143.4

(51) Int. Cl. A61F 2/72; G16H 40/63

(52) U.S. Cl. CPC A61F 2/72; A61F 2002/704; G16H 40/63

(57) ABSTRACT

The invention relates to a method for controlling an orthotic or prosthetic device and an orthotic or prosthetic device, which can be placed on the body of a user and secured, including a joint device having a proximal component and a distal component, which are pivotally mounted on one another about a pivot axis; at least one adjustable actuator which is arranged between the proximal component and the distal component and via which a movement behaviour relating to a pivoting of the proximal component relative to the distal component can be adjusted; at least one detection device for detecting muscle contractions; and a control device which is coupled to the detection device and to the actuator, processes (electrical) signals from the detection unit, and adjusts the actuator according to the signals, wherein the detection device is designed for detecting muscle contractions.

The invention relates to a method for controlling an orthotic or prosthetic device which can be placed on the body of a user and secured thereon, comprising a joint device with a proximal component and a distal component, which are mounted pivotably on each other about a pivot axis, at least one adjustable actuator, which is arranged between the proximal component and the distal component and via which a movement resistance to a pivoting of the proximal component relative to the distal component is adjustable, at least one detection device for detecting muscle contractions, and a control device which is coupled to the detection device and to the actuator, processes signals from the detection device, and adjusts the actuator according to the signals. The invention also relates to such an orthotic or prosthetic device or such an orthosis or prosthesis.

The purpose of prosthetic devices or orthoses is to guide or support the movement of an existing limb or to brace and support a limb. Exoskeletons are also orthotic devices. Orthotic devices for the lower extremity are known in different designs. Those known as knee ankle foot orthoses (KAFO) support both the foot and also the ankle joint and knee joint. The foot is generally placed on a foot plate, one or more lower-leg rails extend parallel to the lower leg, and an orthotic knee joint is provided approximately in the region of the natural knee axis. Securing devices are mounted on one or more thigh rails in order to fasten the orthosis to the thigh. Likewise, securing devices can be provided on the lower leg or the foot plate, so as to be able to fasten the orthosis to the respective leg that is to be managed. As regards the orthotic knee joint, the thigh rail is a proximal component and the lower-leg rail is a distal component, and, as regards an orthotic ankle joint, the foot part is the distal component and the lower-leg rail or the lower-leg rails are the proximal component. Dampers or drives can be arranged as actuators between the respective components, in order to influence movement sequences and to change movement resistances. Either an increased movement resistance can be provided for damping a movement, or a reduced movement resistance can be provided by reducing damping or support by means of one or more motorized or energy-accumulator-operated drives. The same also applies to orthotic devices such as orthoses and exoskeletons for the upper extremity.

Prostheses replace a missing limb and can have an artificial joint. Prostheses of the lower extremity can, for example, have a prosthetic knee joint that is connected to a prosthetic foot, as foot part, via a lower-leg tube as the lower-leg part. A prosthetic ankle joint can be formed between the prosthetic foot and the lower-leg tube. The prosthetic knee joint generally has an upper part and a lower part, which are mounted pivotably on each other via a monocentric or polycentric joint device, such that the upper part is pivotable relative to the lower part about a pivot axis. At least one securing device is arranged on the upper part in order to be able to fasten the prosthesis of the lower extremity to a user, for example for a thigh socket or in the form of a thigh socket. As regards the prosthetic ankle joint, the lower-leg tube is the upper part and the prosthetic foot is the lower part. An actuator can be arranged between the respective upper part and the lower part or the proximal component and the distal component, in order to change the respective movement resistance with respect to flexion and/or extension. As has been stated in connection with an orthosis, the movement resistance can be increased or decreased via the actuator, for example via passive, adjustable dampers or drives such as electric motors or energy-accumulator-operated drives. Besides prostheses for lower extremities, corresponding prostheses are also known for the upper extremities. The above-described features of an orthosis, prosthesis or exoskeleton can also be present on the orthotic and prosthetic devices according to the invention.

DE 10 2008 008 284 A1 discloses an orthopedic knee joint with an upper part and with a lower part which is arranged pivotably thereon and to which a plurality of sensors are assigned, for example a flexion angle sensor, an acceleration sensor, an inclination sensor and/or a force sensor. Depending on the sensor data, the position of an extension stop is determined and correspondingly adjusted.

DE 10 2009 052 887 A1 discloses a method for controlling an orthotic or prosthetic joint with a resistance device and sensors, wherein status information is made available via the sensors during the use of the joint. The sensors detect moments or forces, wherein the sensor data of at least two of the determined variables are linked by a mathematical operation, thereby calculating an auxiliary variable which is used to control the flexion resistance and/or the extension resistance.

In addition, it is known from the prior art to detect muscle contractions of a user via electrodes, for example via surface electrodes or via implanted electrodes, in order to generate control commands on the basis of detected muscle contractions, e.g. to activate or deactivate drives. Such controls based on myoelectric signals are used in particular in prostheses of the upper extremity in order to control complex movements of the prosthetic hand.

Orthotic or prosthetic devices of the lower extremity are for the most part influenced by the movements and loads of the body or of the orthotic or prosthetic device. The accelerations or forces or states that occur are detected by sensors, and a specific damping behavior is set on the basis of the sensor signals. A deliberate influence is possible only by changing the movements or system loads. In active, powered orthotic or prosthetic devices, it is known to displace components relative to one another by a proportional assignment of muscle activity to the drive, for example in order to open or close a hand or to control certain programs via a pattern recognition. Contraction signals are detected in order to switch back and forth between different operating modes.

The object of the present invention is to provide users with an orthotic or prosthetic device and with a method for control thereof, which provide enhanced safety for the user, at the same time with simple implementation and at the lowest possible cost.

According to the invention, this object is achieved by a method and an orthotic or prosthetic device having the features of the independent claims. Advantageous embodiments and developments of the invention are disclosed in the subclaims, the description and the figures.

In the method for controlling an orthotic or prosthetic device which can be placed on the body of a user and secured thereon, comprising a joint device with a proximal component and a distal component, which are mounted pivotably on each other about a pivot axis, at least one adjustable actuator, which is arranged between the proximal component and the distal component and via which a movement resistance to a pivoting of the proximal component relative to the distal component is adjustable, at least one detection device for detecting muscle contractions, and a control device which is coupled to the detection device and to the actuator, processes signals from the detection device, and adjusts the actuator according to the signals, provision is made that the detection device is designed for detecting muscle co-contractions and is arranged on a limb of the user and coupled to the control device, that at least one muscle co-contraction is detected by the detection device, and that the movement resistance of the actuator is changed according to the detected muscle co-contraction. A muscle co-contraction is the tightening of agonists and antagonists for joint stabilization or the simultaneous tightening of two oppositely acting muscles or muscle groups. A muscle co-contraction occurs, for example, when the arm biceps and the triceps are simultaneously tensioned without the forearm moving relative to the upper arm or, depending on the level of contraction, moving more slowly than without tensioning of the antagonist. The same applies to the muscles that extend and flex the lower leg. If oppositely acting muscles or muscle groups are tensioned simultaneously, this is referred to as muscle co-contractions. Muscle co-contractions are used to stabilize joints or limbs, to have maximum control when performing movements, or to stop movements. Muscle co-contractions occur consciously or unconsciously. An unconscious co-contraction occurs, for example, as a reflex in the presence of sudden obstacles, such as a slippery surface, a slippery floor, alarming situations such as explosions, accidents or the like, but also in preparation for potentially dangerous situations, such as when walking along a precipice or across a bridge. The basic tension in the muscular system is increased by a muscle co-contraction. If a corresponding muscle co-contraction is detected via the detection device, for example if the simultaneous tensioning of arm biceps and arm triceps or, for example, of the biceps femoris muscle and the quadriceps femoris muscle are detected by the detection device, then the detection device transmits corresponding signals to the control device, which then changes the movement resistance of the actuator according to the detected muscle co-contractions. The movement resistance can be changed, for example, by the adjustment of valves in a hydraulic damper or in a pneumatic damper. Brakes can likewise be released or activated or other resistances increased or reduced, for example via electrical drives which are switched on or blocked or counteract a displacement of the proximal component relative to the distal component. The basic setting of the actuator preferably remains unchanged, that is to say the resistance curve, once set, or the resistance profile is maintained over a defined movement or a defined load, that is to say that basic resistance behavior remains unchanged over the movement. In selected muscles, in particular in muscles that are responsible for executing the respective movement, or that would be responsible for this if the limb were still present, the muscle activity is detected via the detection device. If a voluntary or involuntary muscle co-contraction is then detected, this points to a special situation that requires a change in the movement resistance. In contrast to prosthesis controls for the upper extremity, muscle co-contractions do not lead to switching to a new operating mode, for example switching over from the operating mode for opening the hand to the operating mode for closing the hand; instead, the same movements continue to be allowed, but at a different resistance level, or the resistance is increased to such an extent that a further movement of the joint is no longer possible under normal load scenarios. It is therefore a variation of an original control behavior, based on the detected co-contraction.

In one embodiment of the invention, the duration and/or the intensity of the muscle co-contraction or of the muscle co-contractions is detected, and the movement resistance is changed according to the duration and/or the intensity of the muscle co-contraction or muscle co-contractions. For example, the movement resistance is changed over a longer period of time in the case of a prolonged muscle co-contraction. Similarly, with a high intensity of muscle co-contraction, the amplitude of change can be increased by comparison with a lower intensity of muscle co-contraction, in order to change the movement resistance in an adapted manner. For example, the movement resistance can be increased upon detection of a muscle co-contraction, which corresponds to the effect of the muscle co-contraction in a healthy limb. For example, if the user of the orthotic or prosthetic device notices a change in the nature of the surface lying ahead, for example a wet area on a smooth floor, the muscle co-contraction causes the movement resistance to increase, for example in a prosthetic knee joint or orthotic knee joint, which gives the user an enhanced feeling of safety, because the joint can be better controlled and oriented by the rest of the muscles. In the case of a prosthesis, the control is then effected via the thigh stump. In active orthotic or prosthetic devices, i.e. devices with motorized drives or other drives, the forces supporting a movement can be reduced, resistances in the drives can be increased or drives can be activated in opposite directions, in order to prevent or impede a pivoting of the distal and proximal components relative to each other.

With increasing muscle co-contraction intensity and/or increasing muscle co-contraction duration, the movement resistance can be increasingly heightened, such that, through the detection of the time profile of the muscle co-contraction intensity and/or muscle co-contraction duration, a proportional change in the movement resistance can take place. If the intensity increases over a co-contraction period, the change is increased. It is likewise possible that, independently of the intensity of the co-contraction, the movement resistance in the presence of a co-contraction is increased for as long as the contraction lasts, i.e. a greater change is effected in the case of a longer co-contraction duration than in the case of a short co-contraction duration. Alternatively or in addition, the resistance can be reduced at the end of a co-contraction and/or upon detection of another co-contraction and/or by an active trigger and/or a voice command. All the methods of change can be applied alone or also together or in any desired combination.

In a development of the invention, provision is made that the resistance is initially increased very quickly, i.e. that the resistance force increases steeply, wherein the resistance is reduced slowly, at least more slowly than the increase, i.e. the decline in the resistance force is less steep.

The change in the movement resistance can be superposed on a preset control program. Modern orthoses or prostheses often have actuators which provide different movement resistances under sensor control, such that, for example in one gait cycle, different flexion and extension resistances are set in the respective gait phases. With position sensors, force sensors, moment sensors, angle sensors and acceleration sensors and also other sensors, it is possible to detect the current state of the prosthesis, orthosis or an exoskeleton and to provide a resistance behavior that is adapted to the state or the movement behavior. This control program can also be maintained in principle when a muscle co-contraction is detected, that is to say the already existing damping concept or drive concept based on the corresponding sensors can basically remain unchanged. Only the resistance level or the duration of different resistances is influenced for the duration of the muscle co-contraction. The resistance profiles over time, in the normal control program, can be changed in amplitude and/or duration by the change in the movement resistance by the detected muscle co-contraction. The overall stability of the respective joint device is thereby increased, and thus also the stability of the orthotic or prosthetic device. If the muscle co-contraction ceases, the preset standard program is activated again.

In principle, it is also possible that the change in the movement resistance is also applied in actuators that do not have another sensor-controlled control process but instead, for example, have a specific damping behavior or movement resistance behavior set to the respective user.

The change in the movement resistance can influence the extent of the movement resistance, that is to say the amplitude of the movement resistance, and/or the duration of the movement resistance, that is to say the time profile over the movement cycle.

In one illustrative embodiment, the muscle contractions and thus also the muscle co-contractions are detected by the detection device as myoelectric signals, pressure signals, inductively generated signals and/or opto-electronically generated signals and are transmitted to the control device. The control device is designed as a data processing device and has a processor or a computer that processes electrical signals. The signals are generated by sensors or detectors that make use of different physical, chemical or biological effects. The detection device can, for example, have surface electrodes for detecting myoelectric signals. Myoelectric signals occur when muscles contract. The advantage of surface electrodes is the ease of handling, the reversible placement, the adaptability to the respective user, and their non-invasive nature. However, surface electrodes may slip and generally have to be arranged with precision in order to provide a sufficient signal quality. Furthermore, it is possible to tap electrical potentials via implants in the muscle and to transmit these via interfaces or wirelessly to receivers, which then transmit the signals in electrical form to the control device. It is likewise possible to position pressure sensors on or in the respective muscles. As a result of the muscle contractions, the shape of the muscle changes, which in turn leads to changes in the pressure, for example on a cuff, brace or a receiving socket for a stump, for example a thigh socket. The sensors can likewise be applied externally to the muscle or limb, for example provided with pretensioning via a belt or via an elastic band, in order to detect the respective pressure changes. Changes in the optical transmissibility can also be utilized to detect muscle contractions. It is possible to accommodate coils in a socket and to insert implanted electrodes in the muscles in a micro-invasive manner. The implanted electrodes are supplied inductively with energy via the coil or the coils in the socket and transmit the recorded data wirelessly or in a wired manner via the coil or coils to an evaluation unit, which then transmits these data to the control device. The muscle activation is basically determined via electrodes, sensors or detectors. In a computing unit, the co-contraction and the intensity and duration thereof are determined from the electrical signals, and the control algorithm is adapted on the basis of this information.

In a variant of the invention, provision is made that the raw signals detected by the detection device, for example surface electrode arrangements, implants, pressure sensor devices, optical sensor devices and/or inductively operating sensor devices, are processed in a pre-processing unit, and only the processed signals are transmitted to the control device. The pre-processing of the raw signals from the respective sensors or detectors takes place, for example, in a microcontroller or in a separate computing unit, which is in the vicinity of or structurally connected to the detection device and the detectors. For example, the pre-processing unit can be arranged on one of the components, for example the proximal component, and can be coupled wirelessly or via a wired connection to the control device. In one embodiment, the pre-processing unit transmits to the control device only the information relevant to the control method, for example the co-contraction intensity and the co-contraction duration, or only the signal that a co-contraction is present, so that the actual control unit, which may also have to perform other control tasks, is not burdened with the processing of raw signals. The control device is thus provided with a processed signal that is easily managed and that can be easily integrated in the already existing control algorithm. The detection device and the pre-processing unit can be constructed as a module and used as an additional component for already existing controls. The module can have its own energy storage. In addition to a high degree of flexibility afforded by the modular design, there is also no negative impact on the battery life of the already existing control device with an additional functionality.

Co-contraction is considered to mean not only the tensioning of agonist and antagonist, but generally the simultaneous tensioning of several muscle groups which meet different or independent functions. For example, if one of the muscles of an antagonist/agonist pair is no longer present, the activation of another muscle or of another muscle group can be defined as an antagonist of the remaining muscle. If, for example, the leg extensor or quadriceps femoris muscle is present, but the leg flexor or biceps femoris muscle is not present, an original co-contraction is not possible, so that, instead of this, the contraction of the abductors and/or the abdominal muscles can be used to generate a co-contraction signal for control.

In the orthotic or prosthetic device which can be placed on the body of a user and secured thereon, comprising a joint device with a proximal component and a distal component, which are mounted pivotably on each other about a pivot axis, at least one adjustable actuator, which is arranged between the proximal component and the distal component and via which a movement resistance to a pivoting of the proximal component relative to the distal component is adjustable, at least one detection device for detecting muscle contractions, and a control device which is coupled to the detection device and to the actuator, processes signals from the detection device, and adjusts the actuator according to the signals, provision is made that the detection device is designed for detecting muscle co-contractions, and the control device is designed for carrying out the method as described above. With such an orthotic or prosthetic device, for example an orthosis, an exoskeleton or a prosthesis, it is possible to voluntarily or involuntarily change the resistance level of the adjustable actuator in a manner appropriate to the situation and to allow an already existing control program to be superposed in order to allow a user to adapt easily and intuitively to an increased need for safety. The joint becomes stiffer, because a relative pivoting of the proximal and distal components is made more difficult or impossible, such that the user acquires better control over the device.

The detection device can be designed as a surface electrode arrangement, as an implant, as a pressure sensor device, as an optical sensor device and/or as an inactive sensor device. The arrangement of the detection device is such that muscle co-contractions can be detected. For this purpose, the respective sensors or detectors are arranged on the corresponding muscles or coupled to them, for example prepared accordingly via implants, such that muscle contractions can be detected, co-contractions can be determined, and corresponding changes can be carried out in the actuator.

In one embodiment, the detection device can be integrated in the proximal and/or distal component. In the case of prostheses, for example, there is a socket for a stump part on the proximal component. Sensors can be permanently installed in the receiving socket and/or arranged in a prosthesis liner. Similarly, recesses or openings for the positioning of electrodes or other sensors can be formed in the socket or another receiving device for a body part, such that different sensors or sensors can be exchangeably and adaptably attached to the respective user at the proximal component. Surface electrodes could be laminated on the inner face of a socket or permanently attached. A separate control device or a pre-processing unit can likewise be integrated in the socket or in the proximal component or fastened thereto, optionally with its own energy supply, in order to process and evaluate the sensor data concerning the respective muscle contractions and to transmit the processed data to the control device, which then accordingly supplies the actuator with control signals for changing the movement resistance.

In one embodiment of the invention, at least one sensor for detecting forces, angles, positions, accelerations and/or moments is arranged on the orthotic or prosthetic device and is coupled to the control device. The respective sensor can be designed as a strain gauge, angle sensor, inertial angle sensor, acceleration sensor or moment sensor. If a sensor can detect several parameters or variables, these parameters or variables can be detected by only a single sensor; otherwise several sensors are necessary for the detection of the different parameters or variables. Via this sensor or via these sensors and the sensor values detected therewith, it is possible to generate signals in order to adapt the actuator, via the control device, to the respective movement state, movement situation, load state and/or user.

The detection device can be coupled to a pre-processing unit in order to be able to identify muscle co-contraction data and to transmit processed data to the control device such that the latter can work more quickly and more efficiently.

The detection device and the pre-processing unit can be designed as a common module and can be fixed to the orthotic or prosthetic device or to the user in order to be coupled to the control device. It is thereby possible for existing orthotic or prosthetic devices, such as orthoses, exoskeletons and prostheses, to be equipped and retrofitted with the detection device and the pre-processing unit.

Illustrative embodiments of the invention are explained in more detail below with reference to the accompanying figures, in which:

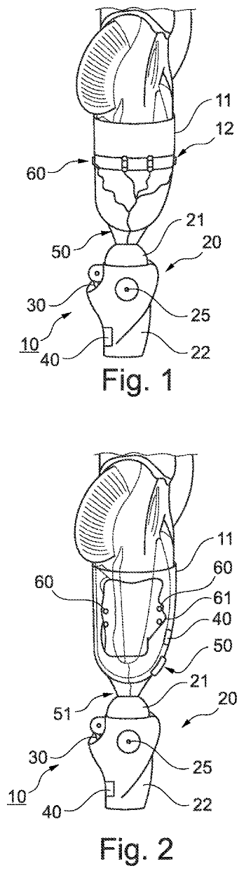

FIG. 1 shows a schematic representation of a prosthetic device in the form of a prosthetic knee joint;

FIG. 2 shows a variant of the prosthetic device according to FIG. 1;

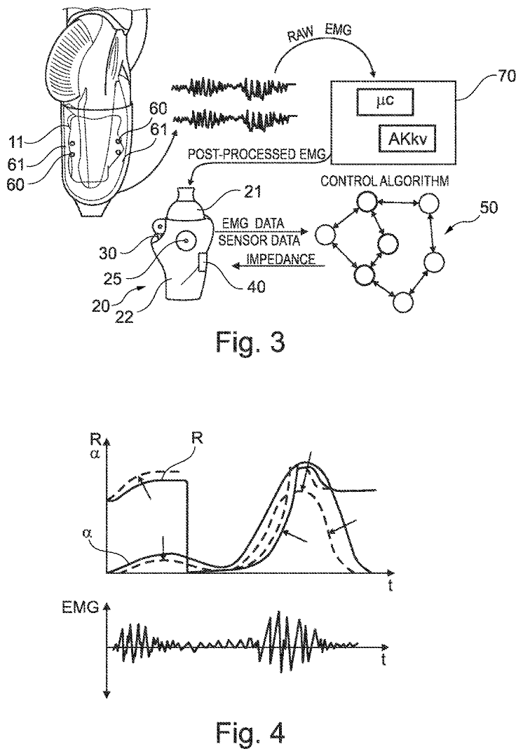

FIG. 3 shows a schematic representation of a variant with a pre-processing unit;

FIG. 4 shows a schematic representation of a change in resistance when walking on the level;

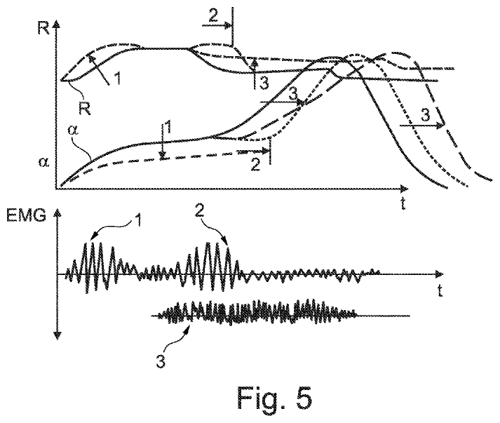

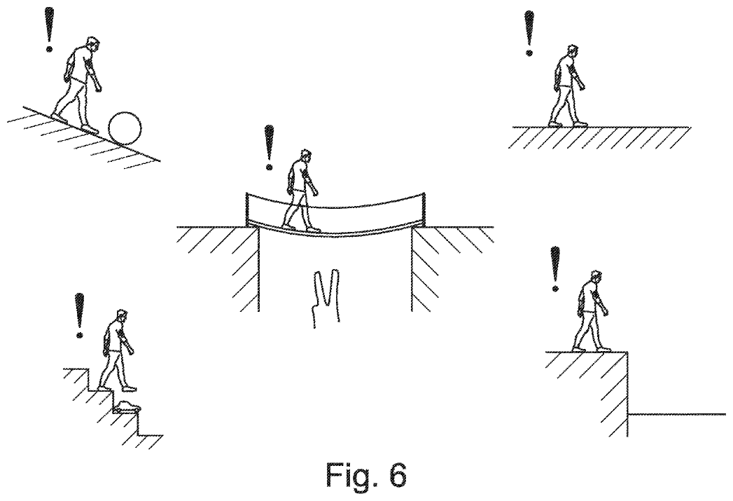

FIG. 5 shows a schematic representation of a change in resistance when walking downward;

FIG. 6 shows an illustration of situations of involuntary co-contractions; and

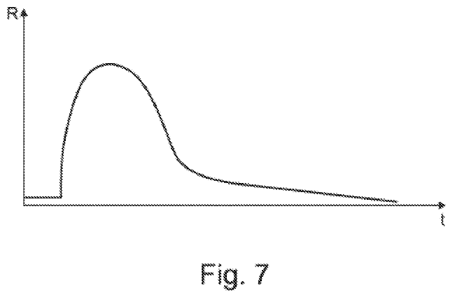

FIG. 7 shows a schematic representation of a resistance curve.

FIG. 1 shows a schematic representation of a prosthetic device 10 in the form of a prosthetic knee joint with a thigh socket 11, which is fastened to a patient. The thigh socket 11 can be fastened to a thigh stump by different mechanisms, for example by suction socket technology, clamping, an osseointegrated fixation, or in other ways. Arranged at the distal end of the thigh socket 11 is a joint device 20 in the form of a prosthetic knee joint, which has a proximal component 21 with connection means for fastening to the thigh socket 11. On the proximal component 21, a distal component 22 in the form of a lower-leg part is pivotably mounted about a pivot axis 25. In the illustrative embodiment shown, the pivot axis 25 is formed as a knee joint axis, while in other applications, for example in a prosthetic ankle joint or in prosthetic or orthotic devices on the upper extremity, other joint axes are accordingly provided. As an alternative to a prosthetic device 10, an orthotic device can also be provided which, instead of replacing a limb, supports a limb that still has a natural joint. In the case of a cross-knee orthosis, a thigh rail is fixed to a thigh and a lower-leg rail is fixed to a lower leg, for example by buckles, straps or shells. The thigh rail and the lower-leg rail are coupled to each other via an orthotic knee joint so as to be pivotable about an axis. In the case of an ankle orthosis, the proximal component is a lower-leg rail and the distal component is a foot part which, in the region of the natural ankle joint axis, is arranged pivotably thereon via a joint. An orthotic device is also understood to mean exoskeletons.

An actuator 30 is arranged between the proximal component and the distal component 22; in the illustrative embodiment shown, the actuator 30 is mainly arranged on the distal component 22 and integrated in a housing. The actuator 30 can be designed as a resistance device, for example as a hydraulic damper, pneumatic damper or magnetorheological damper. It is also possible that the actuator is designed as a motor drive, for example as an electromotive drive, hydraulic drive or pneumatic drive. Even in an embodiment of an actuator 30 as a drive, it can be switched as a resistance device, for example by operating an electric motor in generator mode. Arranged on the proximal component 21 is a jib, which is coupled by a piston rod or a push rod to the resistance device or the drive, i.e. the actual actuator 30, wherein the actuator 30 is connected at the other end of the piston rod or of the push rod to the distal component 22.

The proximal component 21 executes a pivoting movement with respect to the distal component 22; in the position shown, the joint device 20 is in a position of maximum extension. From this position, a flexion movement takes place in which the posterior or rear face of the proximal component 21 is pivoted in the direction of the posterior face of the distal component 22, such that the angle on the posterior face between the two components 21, 22 decreases in the event of flexion and increases in the event of extension. As the flexion angle increases, the knee angle decreases. In order to be able to execute a pivoting movement adapted to the particular walking situation, both in the flexion direction and in the extension direction during walking, the actuator 30 is designed to be adjustable in order to influence the movement behavior during flexion and/or extension. By increasing the flexion resistance, for example, the maximum flexion angle or the minimum knee angle can be set, and, in the case of an extension movement, an increase in resistance can be provided shortly before the position of maximum extension is reached, in order to avoid a hard stop in the extension position. It is likewise possible to configure the actuator 30 to support the movement, that is to say as a drive for initiating or supporting a flexion movement and/or extension movement.

In order to adjust the actuator 30 and to influence the provided resistance or the provided support, the actuator is coupled to a control device 50 which, in the illustrative embodiment shown in FIG. 1, is integrated in the distal end region of the prosthesis socket 11. Data processing devices, connections, contact faces, interfaces and/or an energy store are arranged in the control device in order to process incoming sensor data or data from a detection device 60. In the illustrative embodiment shown in FIG. 1, the detection device 60 is designed as a surface electrode arrangement which is fastened by a belt 12 to the thigh socket 11 and the thigh. The surface electrodes 60 detect myo-electrical signals during the contraction of the thigh muscles and conduct these signals via cable or also wirelessly to the control device 50. In addition, at least one sensor 40 is arranged on the distal prosthesis component 22 in order to detect further data concerning the existing loads, forces, moments, angular positions, accelerations and/or spatial orientations and transmit them to the control device. Although sensors 40 are not required for carrying out the method, they are advantageous as a supplement.

The detection device 60, having a plurality of surface electrodes arranged circumferentially around the stump, allows muscle contractions to be detected via myo-electric signals. As an alternative to an arrangement on a belt 12, the surface electrodes can also be integrated in the prosthesis socket 11. The detection device 60 and the surface electrodes thereon are arranged and designed such that different muscle groups can be detected with regard to their activity. This makes it possible to detect muscles that are responsible for or involved in opposite movements, for example the quadriceps for extension and the leg biceps for flexion, and to detect muscle co-contractions, i.e. simultaneous tightening of muscle groups. Muscle co-contractions do not occur only in muscles or muscle groups that have an antagonistic action. Co-contractions can also occur and be detected when mutually independent muscle groups are tensioned, for example the abdominal muscles together with the hip flexor or the leg flexor.

FIG. 2 shows a variant of the prosthetic device, in which, in addition to a sensor 40 for detecting status information such as forces, moments, angular positions in space or accelerations, a further sensor device 40 is arranged on the thigh socket 11. This sensor device 40 is also coupled to the control device 50, which, in the illustrative embodiment shown, is integrated in the thigh socket 11. The control device 50 has an integrated energy storage unit, for example an accumulator or a battery. In the illustrative embodiment shown, the detection device 60 is formed as a multiplicity of implantable electrodes, that is to say at least two implantable electrodes, which are implanted at different sites in the muscles of the thigh. Muscle activities are detected via these implanted electrodes 60, either by the detection of an electrical potential or by the detection of pressures, temperatures, flow rates, influences of the optical behavior of components or similar. The data detected by the implant can be recorded via a coil arrangement 61, which is arranged on the thigh socket 11 or integrated therein, and transmitted to the control device 50. It is likewise possible that the detection device 60 is designed as a combination of implanted elements and a coil 61 or a plurality of coils 61, in order to detect muscle contractions and, in particular, muscle co-contractions through the inductive effects that occurring in muscle contractions.

Arranged at the distal end of the prosthesis socket 11 is an interface 51 to the joint device 20, via which interface 51 it is possible to transmit the sensor data concerning the muscle co-contractions and, if appropriate, the sensor data from the sensor 40 on the thigh socket 11 to the actuator 30. The sensor data of the sensor 40 on the distal prosthesis component 22 are also transmitted to the control device 50 via the interface 51. The actuator 30 is then actuated and influenced depending on the presence of muscle co-contractions, for example by adjusting valves, throttle cross sections, by activating an electromagnet to influence magnetorheological fluids, by braking a motor and/or by activating a drive.

FIG. 3 shows a further variant of the invention with a basic structure corresponding to that of FIGS. 1 and 2. The thigh socket 11 is disassembled from the proximal component 21, and a mechanical connection of the thigh socket 11 to the joint device 20 can be effected via the pyramid adapter shown. In the illustrative embodiment according to FIG. 3, the detection device 60 is again designed as an implantable electrode arrangement with a coil 61. Alternatively or in addition, it is possible, as shown in FIG. 1, to arrange surface electrodes in recesses within the prosthesis socket 11 at predefined locations and to secure them via the belt 12 or fix them on the inner face of the prosthesis socket 11. By way of the belt 12, which can be designed as an elastic band, the electrodes are held at positions and pressed onto the surface of the skin with sufficient contact pressure. The thigh socket 11 itself is connected electrically conductively to the prosthetic joint 20 in order to transmit the data from the detection device 60 to the joint device 20 and in particular to the actuator 30.

As is shown in FIG. 2, the implanted electrodes 60 can be supplied inductively with energy via the coil 61 on or in the thigh socket 11 and are arranged such that they are located within the field of the coil 61 during use. The data transmission also takes place via the coil 61. According to the embodiment in FIG. 3, the detection device 60 is initially connected to a pre-processing unit 70, to which the raw data are transmitted from the electrodes 60. Instead of via implanted electrodes 60, this can also take place via surface electrodes according to FIG. 1. The raw data are processed in the pre-processing unit 70, for example in a data processing device or in a microcomputer which is arranged, with its own energy supply, in the pre-processing unit 70. The data are processed in a microcontroller, which can be arranged directly on the thigh socket 11, for example integrated in the distal connection piece of the thigh socket 11, in order to forward only the relevant or meaningful information. From the pre-processing unit 70, the processed data are transmitted to the prosthetic knee joint 20, for example via the data connection by means of the electrical coupling of the thigh socket 11 to the proximal prosthetic component 21. The processed raw data of the detection device 60 are transmitted to the control device 50, via which the impedance or the resistance or the drive of the actuator 30 is then controlled. Sensor data of the sensors 40 from the thigh and/or the lower part can also be transmitted to the control device.

The pre-processing unit 70 can be designed together with the detection device 60 as a module that can be arranged on an already existing prosthesis socket 11. In the case of implantable electrodes 60, the electrodes 60 are adapted for example to the coil 61 and the pre-processing unit 70 and have a modular structure.

The detection device 60, 61 and/or the pre-processing unit 70 can be designed to be able to be switched off, such that the prosthesis device 20 can also function without the control device 50 on the basis of the detected muscle co-contractions. The detection device and/or the pre-processing unit 70 are preferably designed to be plug-and-play capable, and therefore a complex coordination process between the individual components is no longer necessary.

The basic principle of the control is similar in all of the illustrative embodiments: the muscle activation is measured and detected via the detection device 60, such as the surface electrodes or implanted electrodes. A computing unit, either in the pre-processing unit 70 or in the control device 50, determines whether there is a muscle co-contraction. The intensity and the duration of the respective muscle contractions is determined. On the basis of the measured muscle contractions (it being possible to store in the control device 50 which simultaneous muscle contractions are regarded as muscle co-contractions), a control algorithm is used to establish whether and how the actuator 30 is activated or deactivated, i.e. whether a change in resistance is to be made or a movement is to be supported. The contraction signals are assigned when the control is set up and adapted to the user. Depending on the position of the respective detection device 60, it is possible to define which muscle or which muscle group causes the corresponding contraction signal.

If a pre-processing unit 70 is assigned to the control device 50 or to the actuator 30 or is connected upstream, the processed data, for example the co-contraction intensity and the duration of the muscle contractions, are transmitted to the joint device 20, such that the joint device 20 has available to it a signal that is easily processed and that can be easily integrated into the control process. As a result, the control effort is reduced and the operating time of the prosthetic device or orthotic device is prolonged, since the signal processing has no negative impact on the running time of the joint device 20 when it is used and, if appropriate, retrofitted with the detection device 60. Particularly if the pre-processing unit 70 can be retrofitted and has its own energy supply, the operating time of the prosthetic joint 20 is not negatively influenced. If appropriate, the total operating time of the orthotic or prosthetic device 10 can be prolonged by simply replacing the pre-processing unit 70 with its own energy supply.

FIG. 4 shows a schematic representation of the course of a movement resistance R and a flexion angle a over time, as a function of the detected muscle contraction signals. A step cycle is shown, starting with the heel strike and ending with the conclusion of the swing phase shortly before the heel touches down. The flexion angle a initially increases, that is to say the knee angle decreases. After the heel has touched down, a so-called stance phase flexion takes place in order to avoid force being transmitted through an extended leg, for example an extended prosthesis or orthosis. The impact movement is thus damped, and the artificial knee joint is allowed to bend slightly. After the foot or the prosthetic foot has been fully set down, a maximum extension takes place during the roll-over to the end of the stance phase, at which a flexion movement already begins, the maximum of which flexion movement is reached in the swing phase when the knee is bent to the maximum. There is then a reversal of movement and an extension of the knee joint. This is shown by the solid line. The resistance R to flexion when walking on a level surface rises to a high level after the heel strike, in order to prevent the knee from bending inadvertently. Even after reaching the roll-over and an onset of stance phase extension, the resistance R remains at a high level, in order then to drop to a low level when the forefoot is loaded, in order to permit flexion for initiating the swing phase. In the further course of walking, the flexion resistance R is increased in the swing phase so that the flexion is not carried out too far, such that an extension of the knee joint can be achieved at the end of the swing phase. Shortly before the reversal of movement is reached, the flexion resistance R is not increased further and, after the maximum flexion angle is reached, it is reduced to the level required for damping the movement during the stance phase flexion. An increased level of flexion damping at the end of the swing phase contributes to the safety of the user, so as to prevent inadvertent bending of the knee in the event of stumbling.

If, as is shown in the lower diagram in FIG. 4, a muscle co-contraction is detected, which can be seen from the high amplitude in both directions, the flexion resistance R is changed, namely increased in the illustrative embodiment shown, as is indicated by the dashed line. A co-contraction can occur involuntarily, for example, when an obstacle is felt or seen or a smooth surface is recognized. Then, when the foot or the prosthetic foot is set down, an increased resistance to bending of the knee joint is already provided, as a result of which the maximum attainable flexion angle a is reduced, which is likewise shown by the dashed line. In the illustrative embodiment shown, the movement influencing takes place through a stronger and earlier increase in the flexion damping in the stance phase flexion. The amount of movement, i.e. the maximum flexion angle a, is thus reduced, and the knee joint feels more compact and stiffer. In a co-contraction during the swing phase, the resistance R can likewise be increased earlier and more strongly, such that the flexion angle increases less quickly and the maximum swing phase flexion is also reduced. The knee joint swings less strongly and therefore comes to full extension much earlier and more quickly. This provides enhanced safety for the user.

A variant of the control method for walking downhill is shown in FIG. 5. Here too, the standard curve of the flexion damping R is shown with the solid line, the knee angle curve a without control via co-contractions is also shown with the solid line. In the standard method, the flexion damping R is increased with the bending in the stance phase until the flexion is blocked. There is then what is called a plateau phase, from which the flexion damping R is reduced to a high level in the middle stance phase, in order to permit further bending, but with a flatter increase in the flexion angle, in order to permit a residual swing phase after heel detachment and relief of the prosthesis and to prepare for the next heel strike. If muscle co-contractions are determined, as shown in the lower diagram in FIG. 5, these resistances can accordingly be increased earlier or maintained at a higher level for longer, such that the knee joint bends later or more slowly and, overall, a higher level of damping against flexion is provided.

In addition to the above-described embodiments of the control of the movement behavior via modified damping, it is possible to influence the movement behavior via other actuators or resistance devices. The ability of a system to counteract a force with a movement is seen as movement influence. This can be done by mechanical elements such as spring elements with a defined stiffness and zero point, damping elements acting in proportion to speed, friction elements and/or masses. Likewise, the movement can be influenced via a motor, a hydraulic or pneumatic damper, spring elements, piezoelectric elements, hydraulic or pneumatic drives or combinations of the stated elements or components. Active drives offer a maximum degree of influence; for example, an electric motor can simulate an elastic behavior and influence the perceived inertia of the joint or of the prosthetic or orthotic device and generate a speed-dependent force.

Examples of situations for voluntary or involuntary co-contractions are shown in FIG. 6, for example when an obstacle appears, when a danger from animals, people or machines is perceived, on smooth surfaces, in the event of sudden dangerous situations, or in unsafe or dangerous environmental conditions. The reflexive muscle co-contraction and also the conscious muscle co-contraction make it possible to influence a control method by muscle activation, wherein a basic pattern of the control method is preferably retained, and only the expressions in terms of strength and duration are changed or influenced. In particular, influencing the movement behavior via muscle co-contractions has the advantage that this can take place both consciously and unconsciously, as a result of which the reflexes of the human being in the perception of the environment can advantageously be utilized to control the orthotic or prosthetic device.

FIG. 7 shows a schematic representation of a resistance curved R plotted over time. If, for example, a co-contraction is determined which appears to make it necessary to increase the resistance to bending, this resistance R is increased very quickly, starting from a basic resistance. After a maximum of the resistance has been reached, for example after the co-contraction has subsided or upon detection of a circumstance that causes the control to reduce the resistance again, a slow reduction in the resistance R is first of all carried out, such that the resistance decreases gently over time. This prevents a rapid drop in resistance from leading to a loss of balance and to unsafe situations for the user.

1. A method for controlling an orthotic or prosthetic device which can be placed on the body of a user and secured thereon, comprising:
a. a joint device with a proximal component and a distal component, which are mounted pivotably on each other about a pivot axis;
b. at least one adjustable actuator, which is arranged between the proximal component and the distal component and via which a movement behavior with respect to a pivoting of the proximal component relative to the distal component is adjustable;
c. at least one detection device for detecting muscle contractions; and
d. a control device which is coupled to the detection device and to the actuator, wherein the control device processes (electrical) signals from the detection device, and adjusts the actuator according to the signals;
wherein the detection device is designed for detecting muscle co-contractions and is arranged on a limb of the user and coupled to the control device, in that at least one muscle co-contraction is detected by the detection device, and in that the movement behavior is changed by the actuator according to the detected muscle co-contraction.

2. The method as claimed in claim 1, wherein the duration and/or intensity of the muscle co-contraction is detected, and the movement behavior is changed according to the duration and/or intensity of the muscle co-contraction.

3. The method as claimed in claim 1, wherein the actuator provides a movement resistance against pivoting, and the movement resistance is increased when a muscle co-contraction is detected.

4. The method as claimed in claim 1, wherein with an increasing co-contraction intensity and/or co-contraction duration, the movement resistance is increasingly heightened, and/or with a decreasing co-contraction intensity and/or co-contraction duration and/or at the end of a co-contraction and/or upon detection of another co-contraction, it is reduced by an active trigger and/or a voice command.

5. The method as claimed in claim 4, wherein the movement resistance is increased more quickly than it is reduced.

6. The method as claimed in claim 1, wherein the change in the movement behavior is superposed on a preset control program.

7. The method as claimed in claim 6, wherein the change influences the extent of the movement influence and/or the duration of the movement influence.

8. The method as claimed in claim 1, wherein the muscle contractions are transmitted from the detection device to the control device as myoelectric signals, pressure signals, inductively generated signals and/or opto-electronically generated signals.

9. The method as claimed in claim 1, wherein at least one sensor detects forces, angles, positions, accelerations and/or moments on the orthotic or prosthetic device and transmits sensor signals to the control device, and the movement behavior is changed on the basis of the sensor signals.

10. The method as claimed in claim 1, wherein the raw signals detected by the detection device are processed in a pre-processing unit and are transmitted in processed form to the control device.

11. The method as claimed in claim 1, wherein the detected muscle co-contractions are checked for plausibility and, in the absence of plausibility, changes in the movement behavior are rejected or reversed.

12. An orthotic or prosthetic device which can be placed on the body of a user and secured thereon, comprising
a. a joint device with a proximal component and a distal component, which are mounted pivotably on each other about a pivot axis;
b. at least one adjustable actuator, which is arranged between the proximal component and the distal component and via which a movement behavior with respect to a pivoting of the proximal component relative to the distal component is adjustable;
c. at least one detection device for detecting muscle contractions; and
d. a control device which is coupled to the detection device and to the actuator, wherein the control device processes signals from the detection unit, and adjusts the actuator according to the signals, and wherein the detection device is designed for detecting muscle co-contractions.

13. The orthotic or prosthetic device as claimed in claim 12, wherein the detection device is designed as a surface electrode arrangement, as an implant, as a pressure sensor device, as an optical sensor device and/or as an inductively operating sensor device.

14. The orthotic or prosthetic device as claimed in claim 12, wherein the detection device is integrated in the proximal and/or distal component.

15. The orthotic or prosthetic device as claimed in claim 12, wherein at least one sensor for detecting forces, angles, positions, accelerations and/or moments is arranged on the orthotic or prosthetic device and coupled to the control device.

16. The orthotic or prosthetic device as claimed in claim 12, wherein the detection device is coupled to a pre-processing unit.

17. The orthotic or prosthetic device as claimed in claim 16, wherein the detection device and the pre-processing unit are designed as a common module.

18. The orthotic or prosthetic device as claimed in claim 16, wherein the detection device and/or the pre-processing unit can be switched off and/or are designed to be plug-and-play capable.

19. The orthotic or prosthetic device as claimed in claim 12, wherein the actuator is designed as a resistance device or drive.

20. An orthotic or prosthetic device which can be placed and secured on the body of a user, the device comprising:
a. a joint device with a proximal component and a distal component mounted pivotably to each other about a pivot axis;
b. at least one adjustable actuator which is arranged between the proximal component and the distal component and via which a movement behavior with respect to a pivoting of the proximal component relative to the distal component is adjustable;
c. at least one detection device in the form of a surface electrode arrangement, implant, pressure sensor device, optical sensor device and/or an inductively operating sensor device for detecting muscle contractions, the detection device being integrated into the proximal and/or distal component of the device; and
d. a control device which is coupled to the detection device and to the actuator, wherein the control device processes signals from the detection unit and adjusts the actuator according to the signals, and wherein the detection device is designed for detecting muscle co-contractions.
